(19) United States
(12) Patent Application Publication
(10) Pub. No.: US 2006/0217739 A1
(43) Pub. Date: Sep. 28, 2006

(54) Phacoemulsification tip

(75) Inventors: Khiun F. Tjia, BM Epe (NL); Mikhail Boukhny, Laguna Niguel, CA (US)

Correspondence Address: ALCON, IP LEGAL, TB4-8, 6201 SOUTH FREEWAY, FORT WORTH, TX, 76134, US

(73) Assignee: ALCON, INC.

(21) Appl. No.: 11/086,038

(22) Filed: Mar. 22, 2005

(51) Int. Cl. A61F 9/00

(52) U.S. Cl. 606/107

(57) ABSTRACT

A belled or flared cutting tip having a plurality of ridges, vanes or fins located in the belled end. Such a feature serves to help break apart lens fragments entering the tip and reduces the potential for clogging of the tip.

### BACKGROUND OF THE INVENTION

This invention relates generally to the field of phacoemulsification and more particularly to phacoemulsification cutting tips.

The human eye in its simplest terms functions to provide vision by transmitting light through a clear outer portion called the cornea, and focusing the image by way of the lens onto the retina. The quality of the focused image depends on many factors including the size and shape of the eye, and the transparency of the cornea and lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an IOL.

In the United States, the majority of cataractous lenses are removed by a surgical technique called phacoemulsification. During this procedure, a thin phacoemulsification cutting tip is inserted into the diseased lens and vibrated ultrasonically. The vibrating cutting tip liquifies or emulsifies the lens so that the lens may be aspirated out of the eye. The diseased lens, once removed, is replaced by an artificial lens.

A typical ultrasonic surgical device suitable for ophthalmic procedures consists of an ultrasonically driven handpiece, an attached cutting tip, and irrigating sleeve and an electronic control console. The handpiece assembly is attached to the control console by an electric cable and flexible tubings. Through the electric cable, the console varies the power level transmitted by the handpiece to the attached cutting tip and the flexible tubings supply irrigation fluid to and draw aspiration fluid from the eye through the handpiece assembly.

The operative part of the handpiece is a centrally located, hollow resonating bar or horn directly attached to a set of piezoelectric crystals. The crystals supply the required ultrasonic vibration needed to drive both the horn and the attached cutting tip during phacoemulsification and are controlled by the console. The crystal/horn assembly is suspended within the hollow body or shell of the handpiece by flexible mountings. The handpiece body terminates in a reduced diameter portion or nosecone at the body's distal end. The nosecone is externally threaded to accept the irrigation sleeve. Likewise, the horn bore is internally threaded at its distal end to receive the external threads of the cutting tip. The irrigation sleeve also has an internally threaded bore that is screwed onto the external threads of the nosecone. The cutting tip is adjusted so that the tip projects only a predetermined amount past the open end of the irrigating sleeve. Ultrasonic handpieces and cutting tips are more fully described in U.S. Pat. Nos. 3,589,363; 4,223,676; 4,246,902; 4,493,694; 4,515,583; 4,589,415; 4,609,368; 4,869,715; 4,922,902; 4,989,583; 5,154,694 and 5,359,996, the entire contents of which are incorporated herein by reference.

In use, the ends of the cutting tip and irrigating sleeve are inserted into a small incision of predetermined width in the cornea, sclera, or other location. The cutting tip is ultrasonically vibrated along its longitudinal axis within the irrigating sleeve by the crystal- driven ultrasonic horn, thereby emulsifying the selected tissue in situ. The hollow bore of the cutting tip communicates with the bore in the horn that in turn communicates with the aspiration line from the handpiece to the console. A reduced pressure or vacuum source in the console draws or aspirates the emulsified tissue from the eye through the open end of the cutting tip, the cutting tip and horn bores and the aspiration line and into a collection device. The aspiration of emulsified tissue is aided by a saline flushing solution or irrigant that is injected into the surgical site through the small annular gap between the inside surface of the irrigating sleeve and the cutting tip.

One phacoemulsification tip that has gained widespread acceptance has a belled or flared distal end. Such a tip is described in U.S. Pat. No. 4,816,018 (Parisi). Such a design allows for larger lens material purchase as well as increased holding force when vacuum is applied to the tip while maintaining a smaller bore in the shaft of the tip. This combination of features increases anterior chamber stability, by reducing sudden outflow from the anterior chamber when the distal end becomes occluded and this occlusion breaks.

Belled or flared phacoemulsification tips do have a tendency to become clogged or occluded because the larger distal end allows larger fragment to enter the tip and become lodged at the base of the bell where the distal tip narrows to the smaller bore of the shaft.

Therefore, a need continues to exist for a belled or flared phacoemulsification cutting tip that resists becoming occluded or clogged.

### BRIEF SUMMARY OF THE INVENTION

The present invention improves upon the prior art by providing a belled or flared cutting tip having a plurality of ridges, vanes or fins located in the belled end. Such a feature serves to help break apart lens fragments entering the tip and reduces the potential for clogging of the tip.

Accordingly, one objective of the present invention is to provide a phacoemulsification cutting tip that resists becoming clogged.

Another objective of the present invention is to provide a phacoemulsification cutting tip having a belled end with a plurality of internal ridges, vanes or fins.

These and other advantages and objectives of the present invention will become apparent from the detailed description and claims that follow.

### BRIEF DESCRIPTION OF THE DRAWINGS

FIG. 1 is a perspective view of a handpiece and control console that may be used with the present invention.

FIG. 2 is a partial longitudinal cross-sectional view of the cutting tip of the present invention.

FIG. 3 is a transverse cross-sectional view of the cutting tip of the present invention taken at line 3-3 in FIG. 2.

### DETAILED DESCRIPTION OF THE INVENTION

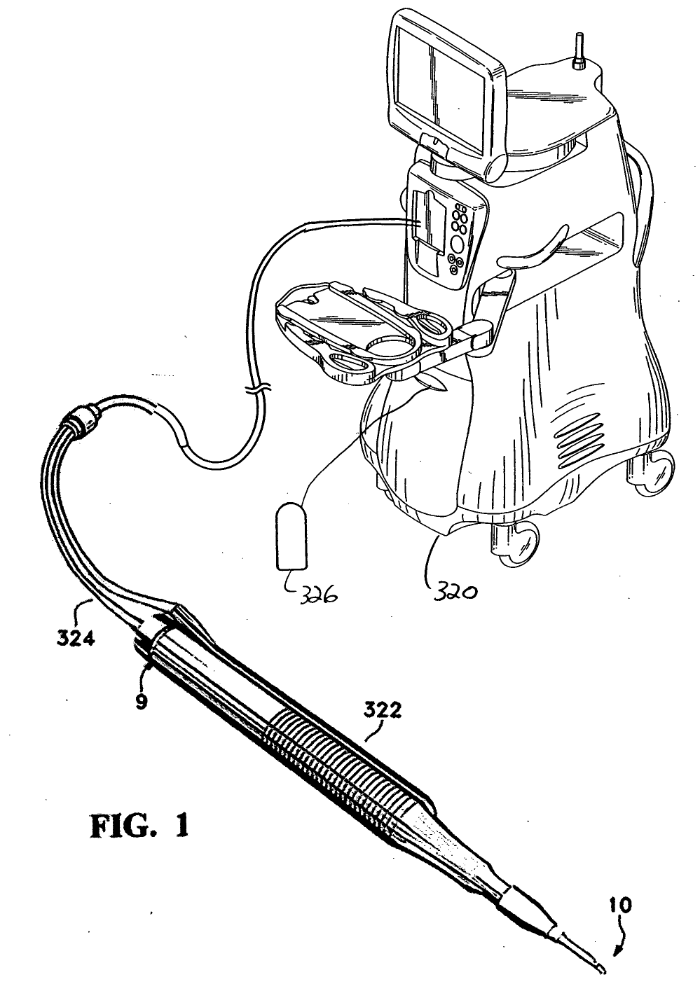

As best seen in FIG. 1, surgical console 320 suitable for use with the present invention may be any commercially available surgical control console such as the INFINITI® surgical systems available from Alcon Laboratories, Inc., Fort Worth, Tex. Console 320 is connected to handpiece 9 through irrigation line 322 and aspiration line 324, and the flow through lines 322 and 324 is controlled by the user, for example, via footswitch 326.

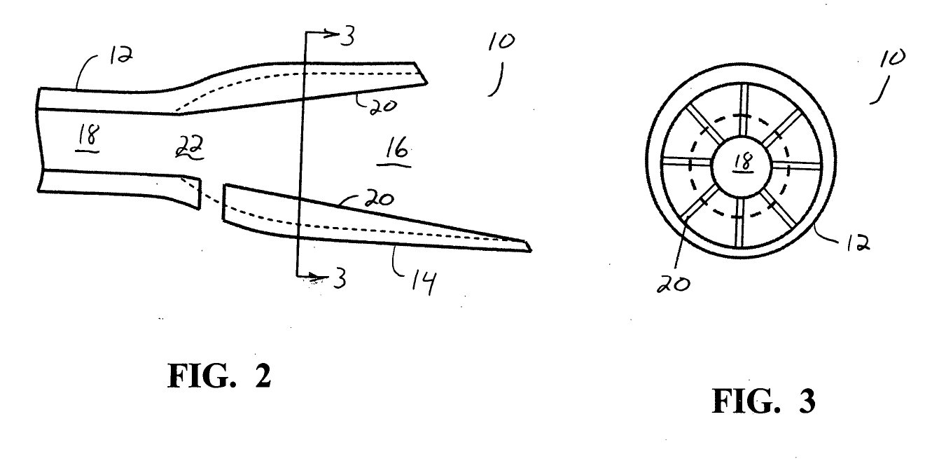

As best seen in FIGS. 2 and 3, phacoemulsification cutting tip 10 of the present invention generally includes shaft 12 integrally formed with flared of belled distal end 14. Cutting tip 10 is preferably made from stainless steel or titanium, but other materials may also be used. Cutting tip 10 preferably has an overall length of between 1.00 inches and 1.50 inches, with 1.325 inches being most preferred. Cutting tip 10 may be formed using conventional metalworking technology and preferably is electropolished to remove any burrs.

Shaft 12 is generally tubular, with an outside diameter of between 0.005 inches and 0.100 inches and an inside diameter of between 0.001 inches and 0.090 inches. Distal end 14 of shaft 12 may be cut square or cut at any suitable angle between 0° and 90°. Distal end 14 has an open mouth 16 that communicated with bore 18 of shaft 12. Projecting internally into mouth 16 from shaft 12 are a plurality of ridges, vanes or fins 20. During ultrasonic vibration of shaft 12, particularly rotational or torsional vibrations of shaft 12, ridges 20 help to break up any material entering mouth 16 of shaft 12, thereby helping to prevent clogging of shaft 12 in throat area 22.

This description is given for purposes of illustration and explanation. It will be apparent to those skilled in the relevant art that changes and modifications may be made to the invention described above without departing from its scope or spirit.

We claim:

1. A phacoemulsification tip, comprising
a) a tubular shaft having a belled or flared distal end, the distal end defining a mouth on the tip; and
b) a plurality of ridges, vanes or fins projecting into the mouth from the distal enter.
